(19) United States
(12) Patent Application Publication
(10) Pub. No.: US 2004/0156924 A1
(43) Pub. Date: Aug. 12, 2004

(54) Vitamin C and calcium ascorbate based dietary supplement products

(76) Inventors: Jonathan Selzer, Cheshire, CT (US); Cornelia D. Vasile, Rocky Hill, CT (US)

Correspondence Address: John L. Cordani, Carmody & Torrance LLP, 50 Leavenworth Street, P.O. Box 1110, Waterbury, CT, 06721-1110, US

(21) Appl. No.: 10/361,375

(22) Filed: Feb. 10, 2003

(51) Int. Cl. A61K 35/78

(52) U.S. Cl. 424/729; 424/757; 424/765; 514/474; 424/774; 424/777

(57) ABSTRACT

A vitamin C based dietary supplement product is revealed with enhanced flavor characteristics. Vitamin C sources are combined with extracts of the fruit of Lo Han and/or extracts from the leaves of Stevia and/or extracts from the leaves of Chinese Blackberry. The resulting product is a pleasant tasting dietary supplement, which is easily absorbed by the body in liquid form, and provides substantial therapeutic effects.

### FIELD OF THE INVENTION

The present invention relates to calcium ascorbate based compositions with improved taste, and to a method for using such compositions as dietary supplements and therapeutic supplements.

### BACKGROUND OF THE INVENTION

The uses of calcium ascorbate as a therapeutic supplement are manifold and well documented in the scientific literature. Current research around the world is substantiating further claims from this form of vitamin C.

Vitamin C is an excellent antioxidant, a characteristic, which plays an important role in many of its physiologically active functions. Vitamin C stimulates the production of collagen and promotes healthy connective tissue. It helps stabilize cartilage and bones and maintains healthy blood vessels. It has a significant anti-inflammatory effect, which may help to reduce some symptoms of degenerative diseases. Vitamin C helps the body to increase its levels of HDL relative to LDL cholesterol. It is required for the biosynthesis of carnitine, a critical player in cellular biochemistry. Vitamin C stabilizes mast cells and plays an important role in histamine and antihistamine physiology, particularly in connection with allergies. It activates folic acid and improves iron absorption. Vitamin C boosts the immune system by enhancing the mobility of white blood cells. It's important role in the synthesis of the neurotransmitters norepinephrine is also well known. Of course, vitamin C also helps prevent scurvy.

Calcium ascorbate has several advantages over regular vitamin C (ascorbic acid). It is absorbed into the bloodstream more quickly and in higher quantities than ascorbic acid. It circulates in the bloodstream twice as long as ascorbic acid and is excreted by the kidneys at a much lower rate. Calcium ascorbate has a pH of 7.0 and thus causes less gastrointestinal discomfort than ascorbic acid, which has a significantly lower pH. Calcium ascorbate produces less oxalic acid in the urine, reducing the risk of the development of kidney stones.

Most dietary supplement products with vitamin C or calcium ascorbate on the market today are in the form of capsules and tablets. In the case of capsules, the vitamin C or calcium ascorbate powders are packaged in either hard gelatin or chemically modified wood cellulose capsules. Tablets are made by compressing the powders together with additives, such as magnesium stearate. Many consumers object to the use of these substances as either (a) animal by-products or (b) chemically derived. Capsules and tablets pose a problem for the very young and the elderly, who may experience difficulties in swallowing such objects. Furthermore, the delivery and absorption of the vitamin C or calcium ascorbate is affected by the use of capsules and tablets, which must be dissolved in the digestive system to release their contents. Studies have shown that many such products will traverse the entire digestive system without being completely dissolved. Thus, not all of the active ingredient of the capsule or tablet is being released for subsequent absorption into the blood stream. On the other hand, drinks, which contain sufficient vitamin C or calcium ascorbate to achieve beneficial effects, can be objectionable because of the intense acidity of vitamin C or the neutral pH (an unpleasant mouth feel) of calcium ascorbate.

Thus, it is an object of this invention to reveal a vitamin C or calcium ascorbate based composition with enough vitamin C activity such that it provides substantial beneficial and therapeutic effects when consumed, yet has a pleasant flavor. It is a further object to this invention to enhance the therapeutic benefits of vitamin C and calcium ascorbate by combining them with flavor enhancing additives, which themselves have beneficial therapeutic properties, but which do not have the problems associated with the use of sugar.

### SUMMARY OF THE INVENTION

The inventors herein propose combining vitamin C, calcium ascorbate or other forms of vitamin C with one or more additives selected from the group consisting of extracts derived from the fruits of Lo Han, extracts derived from the leaves of Stevia, and extracts derived from the leaves of Chinese Blackberry to produce an aqueous drink. The inventors have discovered that dietary supplements prepared in accordance with the foregoing produce a great tasting vitamin C based supplement, which possesses many beneficial therapeutic properties.

### DETAILED DESCRIPTION OF THE INVENTION

The inventors herein propose a dietary supplement comprising:

(a) vitamin C and/or calcium ascorbate and/or dehydroascorbate and;

(b) at least one additive selected from the group consisting of (i) extracts of the fruits of Lo Han, (ii) extracts from the leaves of Stevia, and (iii) extracts from the leaves of Chinese Blackberry.

Additional optional ingredients include xylitol, fructose or other preferably low glycemic sugars, vitamins, minerals, other herbal extracts such as Green Tea polyphenols, citrus bioflavonoids, stabilizers such as Chinese Licorice root extracts, and thickeners or gelling agents. The combination of the foregoing ingredients provides a great tasting dietary supplement product, which possesses the therapeutic properties provided for herein.

The dietary supplement product described herein requires a source of vitamin C. The inventors have found ascorbic acid, calcium ascorbate and/or dehydroascorbate to be useful sources of vitamin C for use in this invention. The ascorbic acid can be of natural source, such as that present in Acerola cherry, cili, rose hips or citrus fruits. It can also be of synthetic origin. Calcium ascorbate and dehydroascorbate are generally manufactured from synthetic ascorbic acid. The concentration of vitamin C in the dietary supplement may range from about 1% to 30% by weight. The most preferred form of vitamin C for this invention is the product Ester-C® (a trademark of Inter-Cal Company), a mixture of several forms of vitamin C. Ester-C® is available from the Inter-Cal Company, located in Prescott, Ariz.

The dietary supplement product described herein also requires at least one additive selected from the group consisting of (i) extracts of the fruits of Lo Han, (ii) extracts from the leaves of Stevia, and (iii) extracts from the leaves of Chinese Blackberry. Preferably, more than one or all of the foregoing additives are present in the composition. The concentration of the foregoing additives in the composition may range in concentration depending upon the concentrations of the extracts themselves. Generally, these extracts are provided as dried powders and if so, the concentration of these additives in the composition of this invention will range from about 0.05% to 6% by weight, but is preferably from about 0.05% to 1% by weight.

Lo Han fruit comes from Momordica grosvenori also called Siraitia grosvenorii. Lo Han is native to the People's Republic of China and Japan and is an edible fruit having an intensely sweet taste. It is reputed to possess healing properties for lung congestion, colds, sore throats, digestive and urinary disturbances, as well as antibiotic and antiseptic properties. Extracts of both fresh and dried Lo Han are available throughout the world, and can be readily purchased. One suitable extract is available from the Fortune Bridge Company, located in New York. Various methods are known for preparing extracts of the Lo Han fruit, such as those methods described in the U.S. Pat. Nos. 6,124,442 and 4,084,010, the teachings each of which are incorporated herein by reference in their entirety.

Stevia (Stevia rebaudiana also called Eupatorium rebaudiana) is also a plant, which is native to the People's Republic of China. Extracts from the leaves of Stevia are commonly known and have been used as a natural sweetener in Asia for many years. Studies have demonstrated that extracts from the leaves of Stevia can have a variety of beneficial therapeutic effects, including antibiotic properties (especially against E. coli), vasodilation properties (especially in the kidney), anti-hypertensive effects, beneficial effects on pancreatic beta cells and enhancing the secretion of insulin. Extracts from the leaves of Stevia are known and can be purchased for use in formulating the compositions described herein. These extracts can be prepared using the same or similar techniques used for preparing extracts for Lo Han as noted above. One suitable extract is available from the Fortune Bridge Company, located in New York.

Extracts from the leaves of Chinese Blackberry (Rubus suavissimus) are also known and can be purchased for use in formulating the compositions described herein. One suitable extract is available from the Fortune Bridge Company, located in New York. The active ingredients in these extracts are believed to comprise diterpene glycosides. Extracts of the leaves of Chinese Blackberry have been used as natural sweeteners. A variety of therapeutic benefits have been ascribed to extracts of the leaves of Chinese Blackberry, including relief from premenstrual syndrome, beneficial effects upon the kidney and other beneficial effects. Extracts of the leaves of Chinese Blackberry can be prepared using the same or similar techniques used for preparing extracts of Lo Han as noted above.

The efficacy of the dietary supplement described herein is improved with the addition of both of the following additives (i) green tea extract and (ii) citrus bioflavonoids. The concentration of the foregoing additives in the composition may range in concentration depending upon the concentrations of the extracts themselves. Generally, these extracts are provided as dried powders and if so, the concentration of these additives in the composition of this invention will range from about 0.05% to 6% by weight, but is preferably from about 0.5% to 5% by weight.

Extracts from the leaves of the tea plant (Camilia sinensis) are well known and can be purchased for use in formulating the compositions described herein. One suitable extract is available and can be purchased from the Fortune Bridge Company located in New York. The active ingredients in these extracts are polyphenols, particularly the catechins. The health benefits of catechins from green tea are multifarious and well documented in the scientific literature. They have been shown to improve cardiovascular health, digestive health, treat and prevent cancer, prevent tooth and gum disease, improve liver detoxification, etc. The extract should have a high catechin content, preferably from 60% to 98% and should be practically free of caffeine. Preferably the extracts of green tea used in this invention contain from 40-90% by weight polyphenols. Suitable extracts can be obtained by hot water extraction from the leaves of the tea plant (Camilia Sinensis). The extract obtained can then be concentrated by evaporation of the water.

Bioflavonoids, which are naturally occurring pigments in fresh fruit, have been shown to work synergistically together with vitamin C to improve absorption and activity. The health promoting effects of bioflavonoids include better eyesight, improved cardiovascular health, increased capillary strength, improved connective tissue structure, better skin appearance and a stronger immune system. Sources of bioflavonoids are well known and it can be purchased for use in formulating the compositions described herein. One suitable extract is available and can be purchased from QCI Company in New York. Bioflavonoids are a class of water-soluble plant pigments. Preferably the bioflavonoid extracts used in this invention contain from 10-80% hesperidin. Suitable bioflavonoid extracts for use in this invention can be obtained by hot water extraction from fruits such as citrus fruits, blueberries, or grapes and other fruits. The extract obtained can be concentrated by evaporation of the water. Preferably, the bioflavonoid extracts used herein are obtained from citrus sources. Other methods of bioflavonoid extraction and invitro synthesis are known and can be used.

As noted, other optional materials may be included in the composition of this invention. Vitamins, minerals or other herbal extracts may be added for particular purposes or effects. Sugars, such as fructose may be added, but are not recommended. Polyol sweeteners, such as xylitol may be added. Preferably, the composition is substantially free of sucrose. Other flavoring agents, such as natural vanilla flavor may also be utilized. Glycerin has also proven to be a beneficial additive.

Stabilizers for the compositions of this invention may also be employed to preserve the flavor and other properties of the product. The inventors have found that extracts from the root of the Chinese Licorice (Glycyrrhiza uralensis) have proven to be excellent stabilizers for the products described herein as well as flavor enhancers. Extracts (typically aqueous extracts) of the root of the Chinese Licorice are known and can be purchased for use in this invention. One suitable extract is available from the Fortune Bridge Company, located in New York. These extracts have been used for their beneficial medicinal properties for years. If used, the concentration of Chinese Licorice extract in the composition of this invention may range from 0.001% to 1.0% by weight.

In preparing the compositions of this invention for use as dietary supplements, the ingredients noted herein are merely combined with purified water in the concentrations noted to form a dietary supplement drink. It is recommended that the consumer of the product ingest one serving of the diluted product per day, such that one receives 400 mg of vitamin C, or 665% of the current RDI (recommended daily intake).

The composition of this invention are further described in the following examples, which should be taken as illustrative only and not limiting in any manner.

### EXAMPLE I

| Component | % by Weight |
|---|---|
| Calcium ascorbate powder¹ | 21.0 |
| Lo Han extract² | 1.2 |
| Chinese Licorice extract³ | 0.1 |
| Natural vanilla flavor⁴ | 0.7 |
| Bioflavonoid⁵ | 3.0 |
| Green tea extract⁶ | 1.0 |
| Glycerin | 33 |
| Water | 30 |

¹Available from Inter-Cal Company
²available from Fortune Bridge Company
³available from Fortune Bridge Company
⁴Available from Flavurence Company in Los Angeles
⁵Available from QCI in New York
⁶Available from Fortune Bridge Company

The foregoing ingredients were blended to form a pleasant tasting dietary supplement, which is believed to exhibit the beneficial therapeutic effects noted herein.

### EXAMPLE II

Example I was repeated, except that Stevia extract powder (available from Fortune Bridge) was substituted for the Lo Han extract powder at the same concentration. The same results were achieved.

### EXAMPLE III

Example I was repeated, except that Chinese Blackberry extract powder (available from Fortune Bridge) was substituted for the Lo Han extract powder at the same concentration. The same results were achieved.

What is claimed is:

1. A composition useful as a dietary supplement, which composition comprises:
a. a source of vitamin C, selected from the group consisting of ascorbic acid, calcium ascorbate, dehydroascorbate, or a combination of the foregoing; and
b. at least one additive selected from the group consisting of (i) extracts of the fruits of Lo Han, (ii) extracts of the leaves of Stevia, (iii) extracts of the leaves of Chinese Blackberry and (iv) combinations of the foregoing.

2. A composition according to claim 1, wherein the composition also comprises at least one material selected from the group consisting of extract of Chinese Licorice root, vitamins, fructose, glycerin, thickeners, gelling agents, xylitol, bioflavonoids, extract of green tea.

3. A composition according to claim 1, wherein the composition is substantially free of sucrose.

4. A composition according to claim 1, wherein the concentration of vitamin C equivalent is from about 10 mg to 2000 mg per serving.

5. a composition according to claim 1 wherein the concentration of the additive(s), on a dry basis, is from about 0.05% to 5.0% by weight.

6. A composition according to claim 2 wherein the concentration of extract of Chinese Licorice root is from 0.001% to 1.0%.

7. A composition according to claim 2 wherein the composition is substantially free of sucrose.

8. A composition according to claim 2 wherein the composition is substantially free of caffeine.
